(19) United States
(12) Patent Application Publication
(10) Pub. No.: US 2020/0046538 A1
(43) Pub. Date: Feb. 13, 2020

(54) KNEE ERGONOMIC DEVICE FOR RESOLVING THE VERTICAL LOADS

(71) Applicant: Giancarlo Pellis, Trieste (IT)

(72) Inventor: Giancarlo Pellis, Trieste (IT)

(21) Appl. No.: 16/658,477

(22) Filed: Oct. 21, 2019

Related U.S. Application Data
(63) Continuation of application No. 12/866,456, filed on Aug. 5, 2010.

(30) Foreign Application Priority Data
Feb. 5, 2008 (IT) TS2008A000001
Jun. 30, 2008 (IT) GO2008A000002

(51) Int. Cl. A61F 5/01

(52) U.S. Cl. CPC A61F 5/0123; A61F 2005/0146; A61F 2005/0165

(57) ABSTRACT

The device consists of a femoral support (1) and a tibial support (2) joined together by means of a lateral hinge and one or two medial hinges. Pins (1.5, 1.6, 1.7, 1.8) and openings (1.9, 1.10) present on the rounded distal end of each femoral arm (1.1, 1.2) and the openings (2.4, 2.5, 2.6) and pins (2.7, 2.8) present on the rounded proximal end of each tibial arm (2.1, 2.2) make up the lateral hinge and a proximal medial hinge. The medial tibial arm can be divided in two parts: a fixed medial tibial arm (2.12) connected to the tibial plate (2.11) and a free-moving medial tibial arm (2.13) secured to the fixed medial tibial arm (2.12) by means of a distal medial hinge. The pins (1.5, 1.6, 1.7, 1.8, 2.15, 2.17) are secured to plates (4, 5). The device can break down the load on mechanical components thus avoiding weight bearing on deficient structures as a result of pathologies or traumas.

### FIELD OF THE INVENTION

The device can be used in the medical sector as a knee brace supporting the vertical loads which are axially transmitted on cartilages.

### BACKGROUND OF THE INVENTION

The knee is the intermediate joint of the lower limb. Its degree of free range allows for a movement of flexo-extension.

The articular mechanics is complex and the type of movement performed is directly related to the opening angle of the knee. If we take the extended lower limb as a starting point, the leg carries out a motion of pure rotation over the first 25-30° of flexion. From that angle on, up to maximal flexion (around 135°), there is a double movement of rolling and gliding. More specifically, after an initial prevalence of rotation of the femoral condyles on the tibial tray, as the flexion movement proceeds, gliding becomes increasingly more progressive and predominant with respect to rolling. Such sliding motion of the joint is known as roto-traslatory.

Moreover, the different anatomical conformation of the lateral and medial compartments of the knee leads—during flexo-extension—to an automatic longitudinal rotation of the tibia vis-à-vis the femur, which is estimated at around 20 degrees by some authors.

Knee braces aim at containing the lateral movements of the knee after suffering a trauma.

Generally speaking, a common knee brace consists of a femoral support and a tibial support. The femoral support includes a lateral femoral arm and a medial femoral arm, which are secured to the thigh by means of straps, parallel to its longitudinal axis. The tibial support comprises a lateral tibial arm and a medial tibial arm, which are secured to the leg by means of straps, parallel to its longitudinal axis.

Each femoral arm is coupled with the respective tibial arm through a hinge at knee level which imposes its movement to the whole joint.

Looking at FIGS. 1 and 2, a more thorough analysis of the knee joint shows that, in the light of the rounded shape of the articular surfaces, there are only two contact points (covered with cartilage) in the joint: one is between the femoral condyle and the tibial tray in the medial compartment (point A), the other being between the femoral condyle and the tibial tray in the lateral compartment (point B).

A healthy knee, therefore, always preserves two contact points between the articular surfaces, their distances remaining perfectly constant.

When traumas and/or pathologies affect the proper functioning of an articular component, such as a ligament, a meniscus or a degenerated articular cartilage, the distance ratios between the articular surfaces are compromised and consequently prevent the entire knee from functioning properly. An injury to the cruciate ligaments does not allow for adequate control of the rolling and gliding movements of the femoral condyles on the tibial trays during flexo-extension. As a result, an anomalous friction of the articular surfaces takes place and the cartilages wear down.

All known devices impose their trajectory to the joint. If the trajectory does not coincide with the physiological one, a traditional device would drag the joint on its own trajectory thereby producing within the joint further tensions which are passed on to the organic structures already weakened by conditions and/or traumas.

This problem has been dealt with by various patents, including the application for European Patent No. 0 361 405, Patent No. WO 84/03433, Patent No. WO 92/15264 and Patent No. WO 97/38759. In particular, Patent No. WO 97/38759 describes a hinge characterized by a roto-traslatory motion. The hinge consists of two plates, which can freely rotate one on the other, each provided with a femoral arm and a tibial arm. The first plate has two openings, one placed centrally, the other peripherally. The central opening has a rectangular shape, with preferably rounded extremities. Its proximal extremity corresponds with a hole situated at the centre of the plate itself. The central opening starts from this central hole and proceeds towards the periphery of the plate along the “a” axis which coincides with the symmetry axis of the tibial arm. An extremity of this second opening is situated on a “b” axis, perpendicular to the “a” axis at a distance “I” from the centre of the above-mentioned central hole. The other extremity of the second opening is placed at 130-140 degrees vis-à-vis the axis which originates the first extremity. The peripheral opening is specifically shaped: initially, in the first 25-30 degrees starting from the above-mentioned axis which generates the first extremity, it is a circumference whose centre coincides with the centre of the plate, its radius being equal to “I”; subsequently, in the remaining 105-110 degrees, it is a spiral heading back towards the centre of the plate. The sequence of points which make up the longitudinal axis of the spiral is obtained from the sequence of points of the extremity of a segment having an “I” length, whose second extremity moves along the longitudinal axis of the central opening, from the centre of the plate to the outside.

The second plate has two pins, placed at a distance equal to “I”, each with its longitudinal axis orthogonal to the rotation surfaces of the plates. The first pin is situated in a central position on the second plate and slides in the central opening of the first plate. The second pin is situated in a peripheral position and slides in the peripheral opening. They are both through pins and are equipped with a distant constraint that prevents disjunction of the two plates. The axis of the central pin is defined as “c” axis and corresponds with the horizontal intercondylar axis around which the initial rotation of the knee develops.

The resulting hinge performs a roto-traslatory motion. If we assume the extended leg as a starting point, in the first 25-30 degrees the hinge carries out a purely rotatory movement imposed by the second pin which slides in the circular section of the peripheral opening, similarly to the rotation described by the knee in this arch-shaped movement. As flexion proceeds, the rotatory motion is accompanied—in an increasingly progressive way—by a sliding motion of the second plate vis-à-vis the first one, when the second pin travels along the spiral-shaped part of the peripheral opening, similarly to what happens between the femoral condyles and the tibial trays in the 25-30° to 135° range.

The hinge described so far is functional for a theoretical knee. However, the length of the cruciate ligaments, their sizes and their insertion points—which characterize the articular surfaces—are different from one person to another. In order to study the true articular profile of a knee, it would be necessary to submit each individual to X-rays or to experimental measurements of the knee-malleolus distance; as a result, the spiral-shaped path followed by the hinge should be adjusted to that of the movement of the knee in each individual subject.

Patent No. WO 97/38759 does solve the problem of the typical roto-traslatory movement of the knee and is particularly indicated to restrain the knee laterally and antero-posteriorly in case of prevalently ligamentous deficiency. However, it does not take into account the longitudinal rotation of the tibia and the possibility of providing vertical support to the knee in case of traumas or degeneration of menisci and articular cartilages.

### SUMMARY OF THE INVENTION

The object of the present invention is to provide a knee device capable of tracking all the movements performed by the knee during flexo-extension.

The device is also designed to break down the load determined by the bodyweight on various mechanical organs, parallel to the knee itself, so as to prevent the load itself from bearing on organic structures weakened by pathological and/or traumatic causes.

The device consists of a femoral support and a tibial support.

The femoral support comprises a lateral femoral arm and a medial femoral arm, parallel to one another and to the longitudinal axis of the thigh. They are joined together by means of a rigid or semirigid arch-shaped plate and straps passing through linear openings situated on the femoral arms. The femoral arms could also be joined together by straps alone.

The tibial support consists of a lateral tibial arm and a medial tibial arm, parallel to one another and to the longitudinal axis of the leg. They are joined together by means of straps which pass through linear openings situated on the tibial arms. The tibial arms could also be joined together by means of an arch-shaped plate and the afore-mentioned straps. Such tibial plate could also be secured to each tibial arm by a fastener.

The distal ends of the femoral arms and the proximal ends of the tibial arms are articulated to each other by means of at least two hinges, one placed laterally and the other medially to the knee. The distal ends of each femoral arm and the proximal ends of each tibial arm—which make up the hinge—are shaped as rounded plates.

The rounded distal end of each femoral arm accommodates four screwed pins and two openings. The first pin is placed centrally on the distal end along the “a” axis which coincides with the axis of longitudinal symmetry of the femoral arm itself. The second pin is placed peripherally on the end of the femoral arm, at a distance “I” with respect to the central pin, along a “b” axis perpendicular to the “a” axis. The axes “a” and “b” ideally divide the distal end of the femoral arm into four quadrants: the first one oriented towards the foot and the posterior part of the leg; the second one oriented towards the foot and the anterior part of the leg; the third one oriented towards the root of the lower limb and the posterior part of the leg; the fourth one oriented towards the root of the lower limb and the anterior part of the leg.

The third pin is placed proximally with respect to the afore-mentioned pins, in the quadrant oriented towards the root of the lower limb and the posterior part of the leg. It is situated at a distance “r” with respect to the central pin. The “d” axis which passes through the centre of the proximal pin and the centre of the central pin deviates by a few degrees from the “a” axis. The peripheral edge of the third pin is tangential to the “a” axis itself.

The “d” axis continues towards the peripheral distal edge of the femoral arm.

Along such axis—at a distance “r” from the central pin—is situated the fourth pin, diametrically opposed to the proximal pin vis-à-vis the central pin. The peripheral edge of the distal pin too is tangential to the “a” axis. The distal pin is situated in the quadrant oriented towards the foot and the anterior part of the leg.

The first opening is located near the peripheral edge of the rounded distal end of the femoral arm and is extended in the two quadrants oriented towards the posterior part of the leg. At the origin of this first opening, one of its extremities is situated at a distance “r” from the central pin along the “d1” axis which is perfectly symmetrical to the “d” axis vis-à-vis the “a” axis. The edge of the first extremity of the first opening is tangential to the “a” axis and to the peripheral edge of the distal pin. The first opening, after describing an initial arc of circumference, turns into a spiral which gets near the rounded distal end of the femoral arm.

The second opening, diametrically opposed to the first one with respect to the “a” axis, is located in the two quadrants oriented towards the anterior part of the leg. At the origin of this second opening, one of its extremities is situated at a distance “r” from the central pin and deviates by a few degrees from the longitudinal axis “a” of the femoral arm. The edge of the first extremity of the second opening is tangential to the “a” axis and to the peripheral edge of the proximal pin. The second opening, after describing an initial arc of circumference, turns into a spiral which—contrary to the first opening—gets away from the centre of the rounded distal end of the femoral arm.

The proximal end of each tibial arm is provided with three openings and two pins. The first tibial opening has a rectangular shape and rounded extremities. Its proximal extremity is obtained by creating a hole at the centre of the proximal extremity of each tibial arm and then proceeding towards the foot along the “a1” axis of longitudinal symmetry of the tibial arm.

The second opening is located peripherally on the proximal end of each tibial arm and is extended for 130°-140°. The first extremity of this peripheral opening is situated on a “b1” axis, perpendicular to the “a1” axis, at a distance “I” from the centre of the above-mentioned central hole from which the first opening originates. The other extremity of the second tibial opening is placed at 130-140° with respect to the axis which originates the first extremity. The peripheral opening, after describing an initial circumference whose centre coincides with that of the proximal extremity of the central opening and whose radius is equal to “I”, turns into a spiral which returns towards the centre of the plate.

The “a1” and “b1” axes ideally divide the proximal end of each tibial arm into four quadrants; the first one is oriented towards the foot and the posterior part of the leg; the second one is oriented towards the foot and the anterior part of the leg; the third one is oriented towards the root of the lower limb and the posterior part of the leg; the fourth one is oriented towards the root of the lower limb and the anterior part of the leg. The central opening is placed in the two quadrants oriented towards the foot, the second tibial opening is placed in the two quadrants oriented towards the root of the lower limb.

The third tibial opening, which is extended for 130°-140°, has a first semicircular extremity that is placed at a distance “r” from the intersection of the “a1” and “b1” axes in the quadrant oriented towards the foot and the anterior part of the leg, and deviates by a few degrees from the “a1” axis. The edge of the extremity of the third tibial opening is tangential to the “a1” axis. The third tibial opening, after describing an initial arc of circumference, turns into a spiral which gets near the rounded proximal end of the tibial arm.

The first tibial pin is situated distally vis-à-vis the central opening, at a distance “r” from the intersection of the “a1”, “b1” axes, in the quadrant oriented towards the foot and the posterior part of the leg. The “d2” axis which passes through the centre of the pin and the intersection of the “a1”, “b1” axes deviates by a few degrees from the “a1” axis. Its peripheral edge is tangential to the “a1” axis itself and to the first extremity of the second peripheral tibial opening.

The second tibial pin is diametrically opposed to the first tibial pin with respect to the “a1” axis; consequently the axis which passes through the centre of the pin itself and the intersection of the “a1” and “b1” axes deviates by a few degrees from the “a1” axis, though in the opposite direction. The peripheral edge of the second tibial pin too is tangential to the “a1” axis. The second tibial pin is placed in the quadrant oriented towards the root of the lower limb and the anterior part of the leg.

The peripheral edge of the end of the tibial arm is extended in the quadrants oriented towards the posterior part of the leg and is shaped in such a particular way that its initial section—extending in the posterior quadrant oriented towards the root of the limb—describes a circumference arc. The successive section of the peripheral edge—extending in the posterior quadrant oriented towards the foot—is shaped as a spiral that tends to get away from the centre of the plate. The peripheral surface of the proximal pin of the femoral arm remains in constant contact with the peripheral edge of the tibial arm during the roto-traslatory movement of the latter vis-à-vis the femoral arm.

The longitudinal axis of the central pin of the lateral hinge and the longitudinal axis of the central pin of the medial hinge are coaxial. Such axis coincides with the “c” axis which passes through the femoral condyles and around which the phase of initial rotation of the knee takes place.

The medial tibial arm can be divided in two parts: a fixed medial tibial arm (fastened to a tibial plate) and a free-moving medial tibial arm which pivots on the fixed arm itself by means of a second medial hinge. The tibial plate is arc-shaped, is made of rigid material with a good harmonic response and tracks the anterior part of the leg. The mobile medial tibial arm consists of a rectilinear plate extending between the medial femoral arm and the fixed medial tibial arm. The medial femoral arm of the femoral support is secured to the proximal end of the free-moving medial tibial arm by means of the above-mentioned medial hinge. The distal end of the mobile medial arm is provided with a hole (situated along the symmetry axis of the mobile arm itself), engaged by a pin placed at the intersection between the symmetry axis of the tibial plate and the symmetry axis of the fixed medial tibial arm. An arc-shaped opening is located along the symmetry axis of the mobile medial tibial arm, in a proximal position with respect to the hole. Such opening accommodates a second pin, which is secured to the fixed medial tibial arm and is situated proximally vis-à-vis the pin along the symmetry axis of the fixed medial tibial arm itself. These elements form a second medial hinge.

More arc-shaped openings and more holes can be made on the distal end of the free-moving medial tibial arm along its symmetry axis. The centre of a semicircular extremity of the openings is placed along the same symmetry axis.

The lateral tibial arm too can be divided into a proximal lateral tibial arm and a distal lateral tibial arm. The proximal lateral tibial arm has an elongated shape and the distal lateral tibial arm is fastened to the tibial plate. At least three equidistant holes are made on the prolongation of the proximal lateral tibial arm and along its symmetry axis. The distal lateral tibial arm accommodates at least two threaded holes, coaxial to at least two holes present on the proximal lateral tibial arm. Two screws pass through the holes of the proximal lateral tibial arm and are secured to two threaded holes of the distal lateral tibial arm.

The pins of the distal ends of the femoral arms and of the fixed medial tibial arm are secured to a plate external to each hinge by means of fasteners. In each plate there are holes placed coaxially with the pins of the femoral arms and of the fixed medial tibial arm. It is through such holes of each plate that the free ends of the pins themselves or the afore-mentioned fasteners engaged to the pins do pass, thereby securing each plate to each femoral arm and to the fixed medial tibial arm, and preventing their disjunction. The fasteners could be screws for engaging the threaded holes of the pins or rivets with their free end engaging in the holes of the pins or nuts that engage the external thread of the pins. The height of the pins is greater than the thickness of the tibial arm.

The present device is capable—even in weight-bearing conditions—of keeping the articulation surfaces well spaced, though submitted to the weight of the body that tends to compress them on one another.

The knee support, while tracking the knee in its motion of antero-posterior flexo-extension, at the same time prevents the lateral and antero-posterior movements of the knee determined by deficient articular structures following a trauma.

The device allows the knee to carry out an ergonomic movement which adequately tracks the physiological one, particularly in the light of the longitudinal automatic rotation of the tibia, contrary to the movement imposed by the best known devices.

### BRIEF DESCRIPTION OF THE DRAWINGS

Further features of the present invention will emerge from the description of an embodiment, preferable but not exclusive, of the device, illustrated in the attached drawings by way of example and not limitation:

FIG. 1 is a front elevation view of a lower limb;

FIG. 2 is an elevation view from above of the tibial trays along the transversal plane of the knee, showing the points of contact with the femoral condyles and the “c” axis;

FIG. 3 is a front elevation view of the knee;

FIG. 4 is an elevation view from above of the tibial trays along the transversal plane of the knee, showing the points of contact with the femoral condyles;

FIG. 5 shows the lower limb in extended position with the indication of the longitudinal axis, transversal axis and front plane;

FIG. 6 is an elevation view from above of the skeletal portion of the knee, showing the points of contact between the articulation surfaces of the knee and the front elevation view;

FIG. 7 shows the lower limb in flexed position with the indication of the front plane of the thigh and the front plane of the leg;

FIG. 8 is an elevation view from above of the skeletal portion of the knee flexed at 90 degrees, showing the contact points between the articulation surfaces of the knee, the front elevation view of the leg and the front elevation view of the thigh;

FIG. 9 is a lateral elevation view of a first preferred embodiment of the present invention, secured to the wearer's limb from a medial perspective;

FIG. 10 is a front elevation view of the device shown in FIG. 9, secured to the wearer's limb viewed frontally;

FIG. 11 is a lateral elevation view of a femoral arm of the device;

FIG. 12 is a lateral elevation view of a tibial arm of the device;

FIG. 13 is a lateral elevation view of the same femoral arm shown in FIG. 11 with some schematic representations of construction;

FIG. 14 is a lateral elevation view of the same tibial arm shown in FIG. 12 with some schematic representations of construction;

FIGS. 15, 16, 17 and 18 show the movements of a hinge of the device;

FIG. 19 shows the distribution of load on the leg when using the present device;

FIG. 20 is a perspective view of the femoral support of the device in a second preferred embodiment;

FIG. 21 is a perspective view of the free-moving medial arm of the device in a second preferred embodiment;

FIG. 22 is a perspective view of the tibial support of the device in a second preferred embodiment;

FIG. 23 is a lateral elevation view of the device in a second preferred embodiment, secured to the wearer's limb viewed medially;

FIG. 24 is a lateral elevation view of the device in a second preferred embodiment, secured to the wearer's limb viewed laterally;

FIG. 25 is a front elevation view of the device in a second preferred embodiment, secured to the wearer's limb viewed frontally;

FIG. 26 is a front elevation view of the free-moving tibial arm;

FIG. 27 shows the device in a second preferred embodiment, without the covering plates, in a position corresponding to that adopted when secured to the leg in the extended position;

FIG. 28 shows the same device, without the covering plates, in a position corresponding to that adopted when secured to the leg on a 15-degree flexion;

FIG. 29 shows the same device, without the covering plates, in a position corresponding to that adopted when secured to the leg on a 35-degree flexion;

FIG. 30 shows the same device, without the covering plates, in a position corresponding to that adopted when secured to the leg flexed above 90 degrees;

FIG. 31 is a front elevation view of a modified free-moving tibial arm;

FIG. 32 is a lateral elevation view of a modified proximal lateral tibial arm, separated from the distal lateral tibial arm;

FIG. 33 is a lateral elevation view of a modified distal lateral tibial arm;

FIG. 34 shows the distribution of forces on a hinge in case of chondropathy;

FIG. 35 shows the distribution of forces on a hinge in case of injury or trauma to the posterior cruciate ligament.

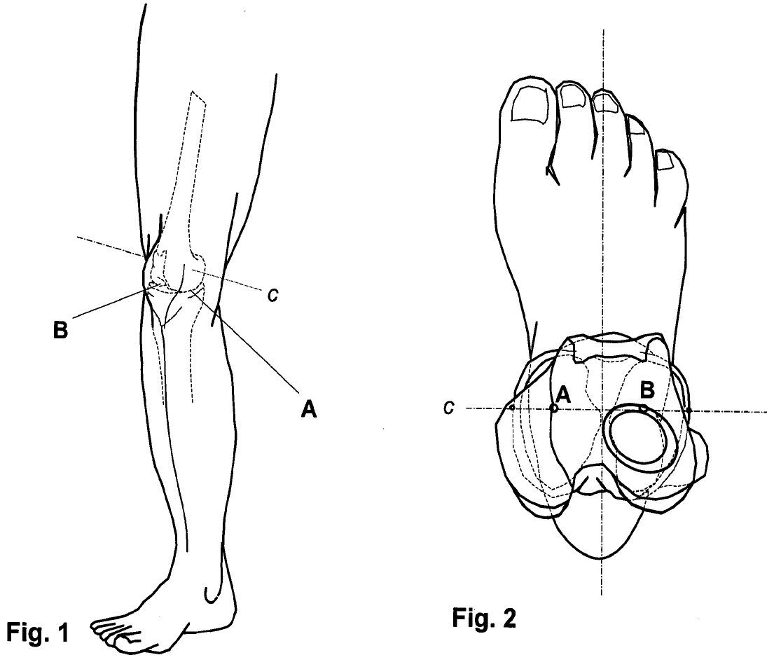

### DETAILED DESCRIPTION OF TWO PREFERRED EMBODIMENTS

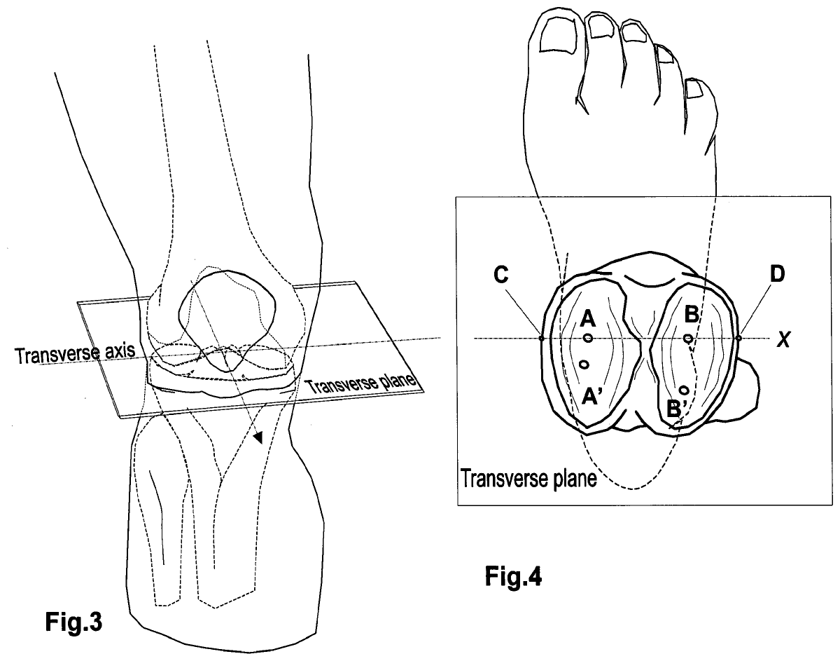

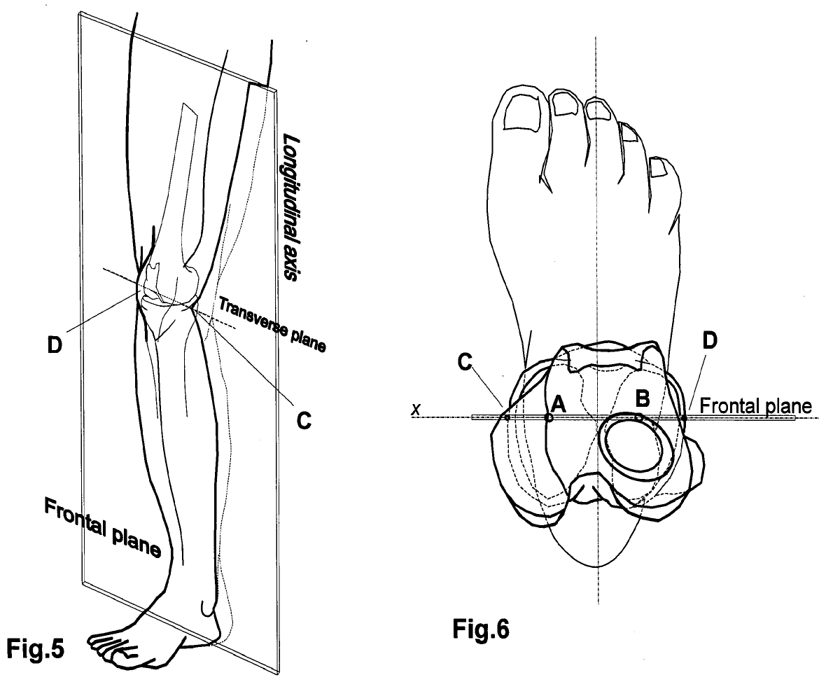

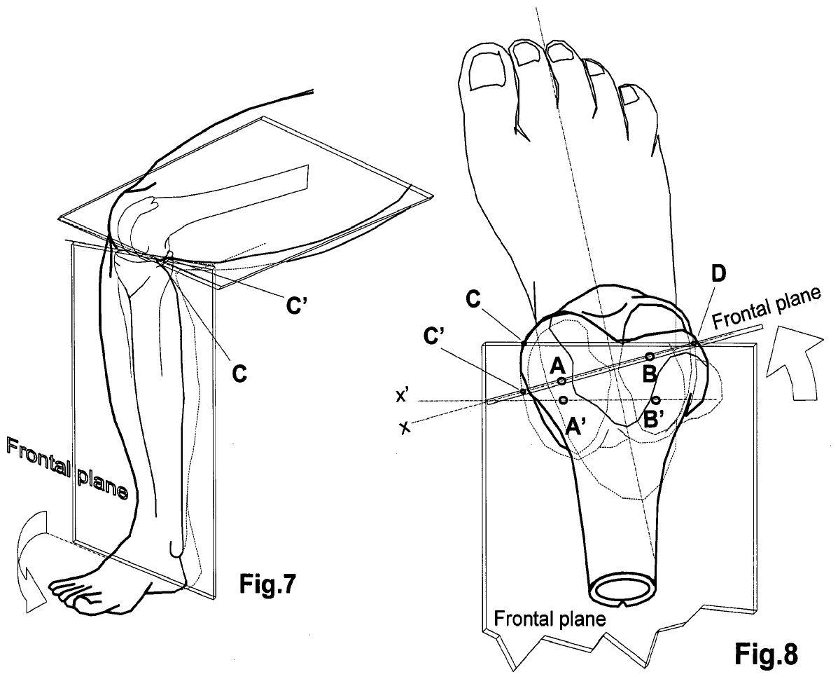

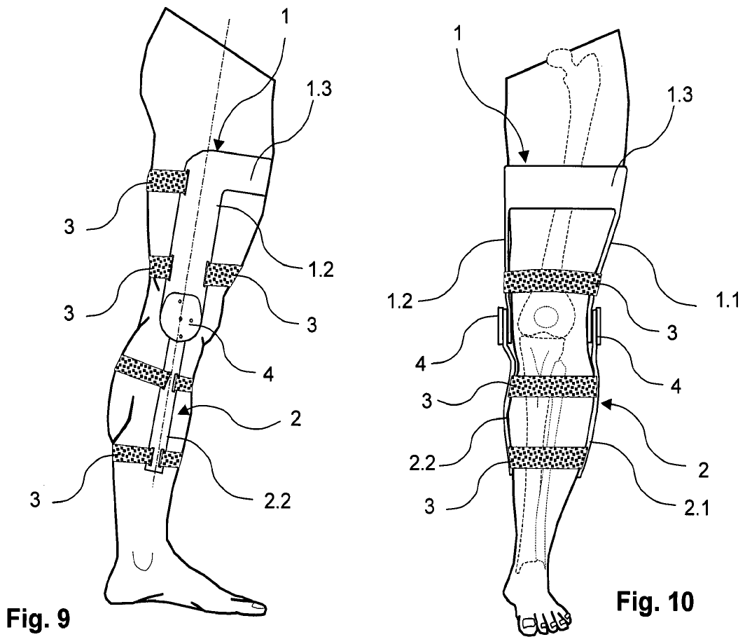

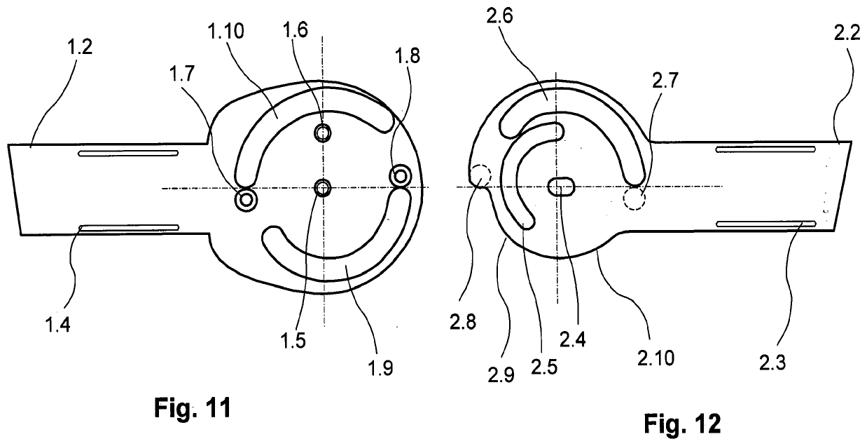

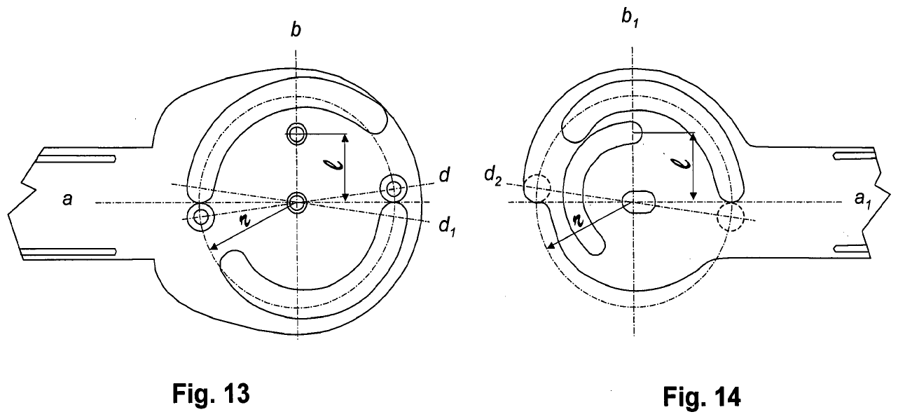

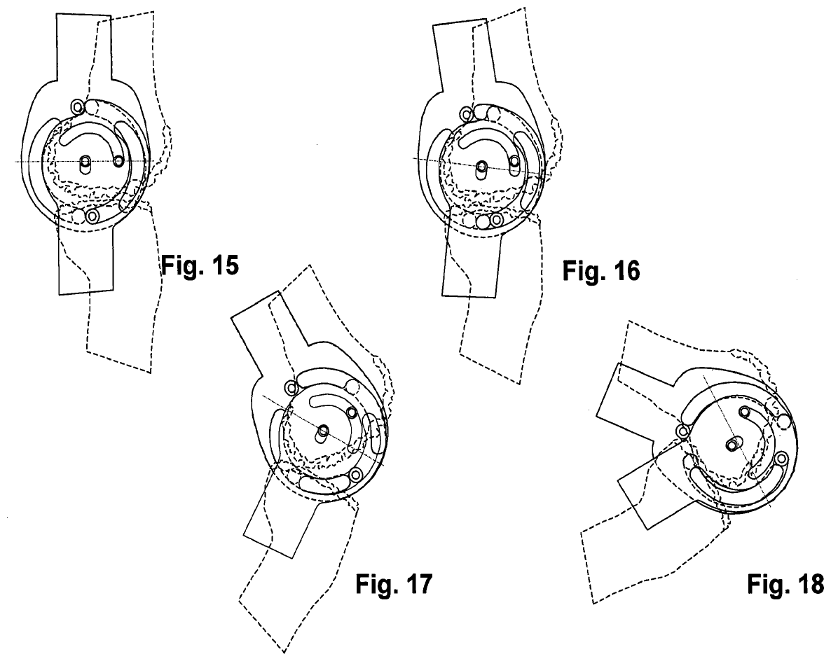

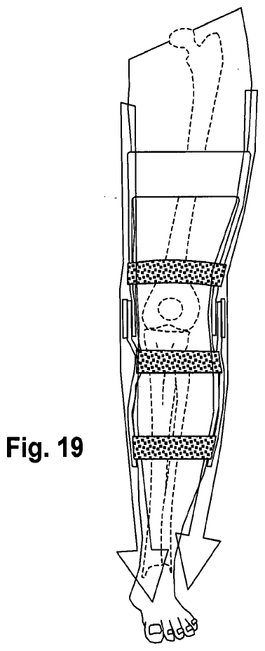

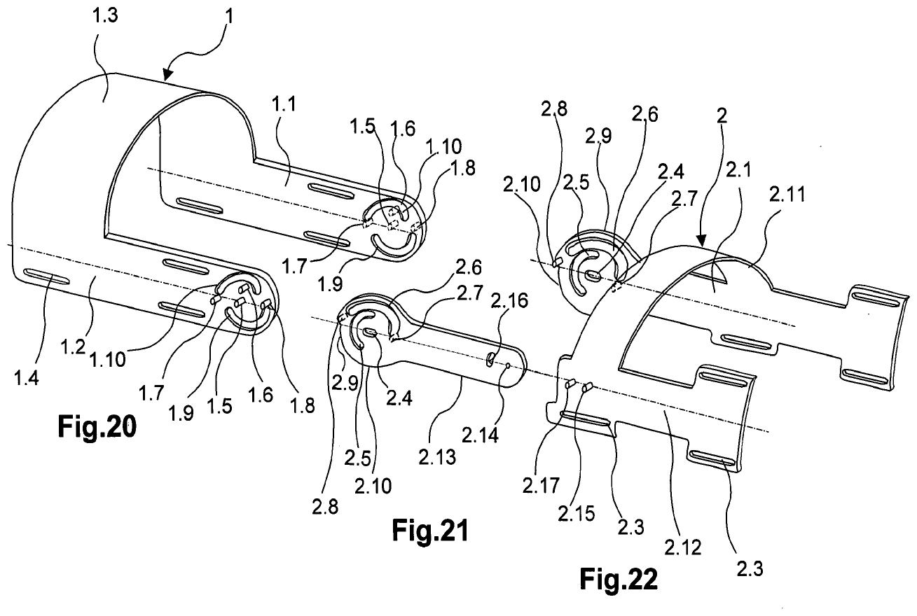

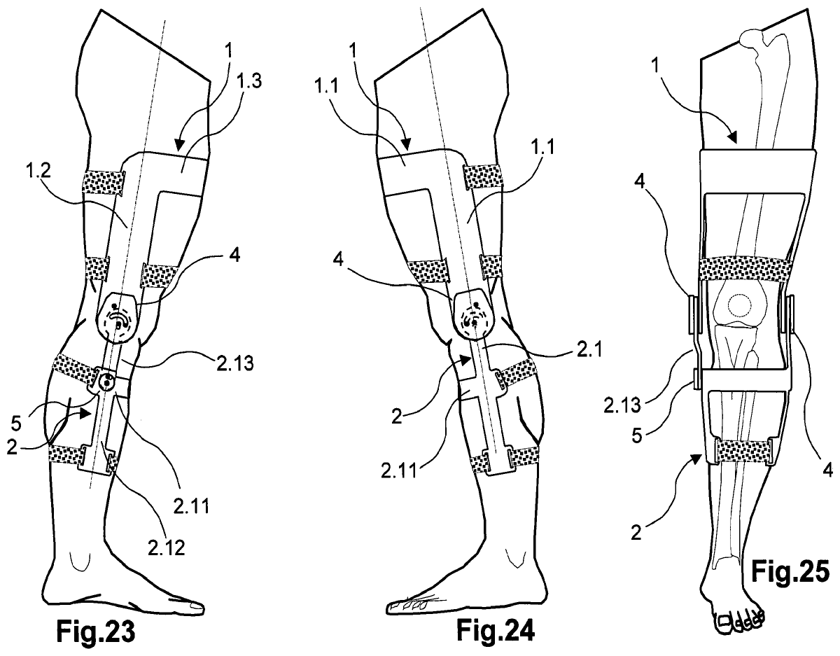

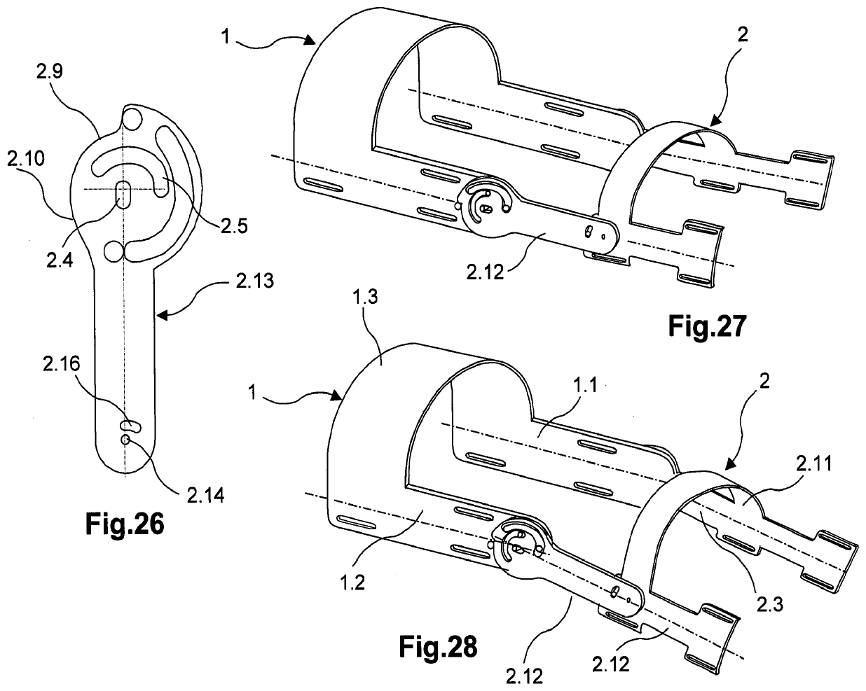

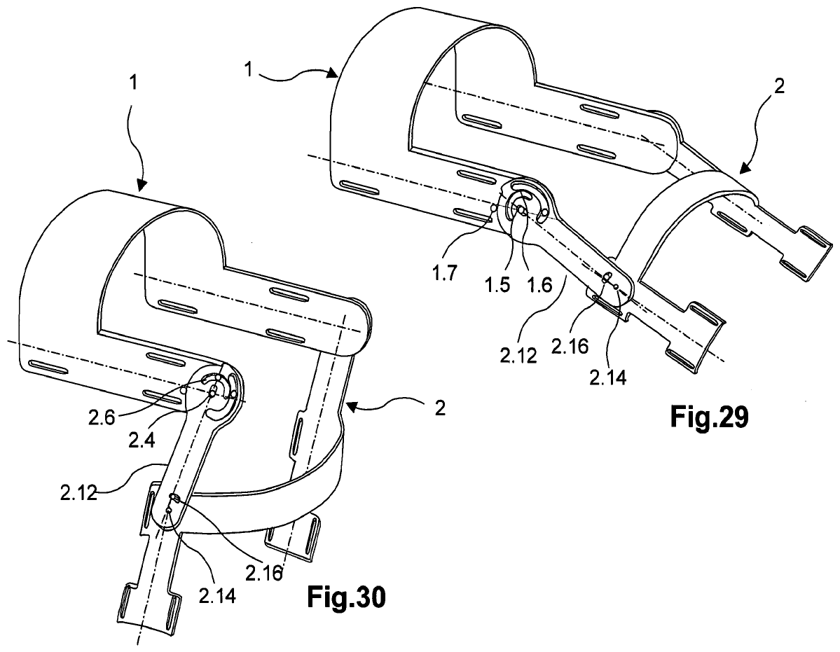

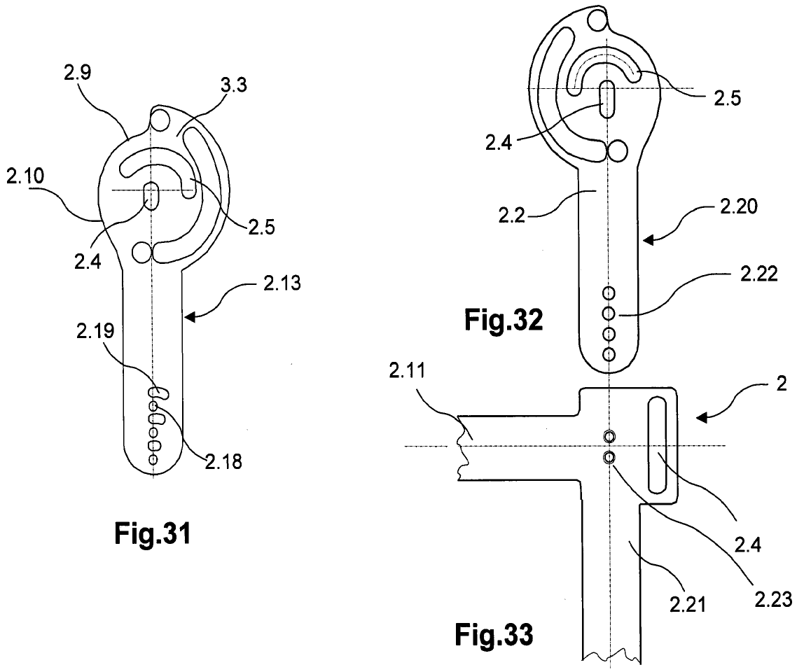

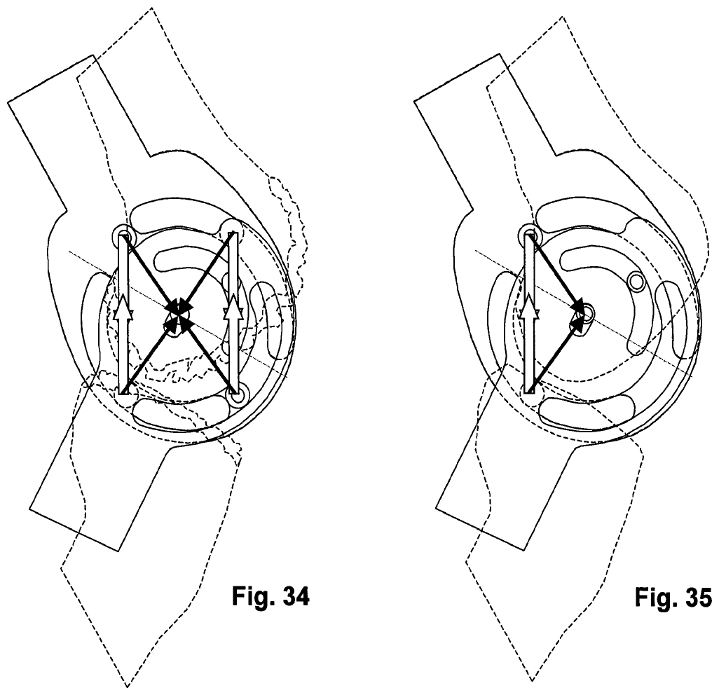

As shown in FIGS. 3 to 8, the first preferred embodiment of the present invention consists of a femoral support 1 and a tibial support 2.

The femoral support 1 comprises a lateral femoral arm 1.1 and a medial femoral arm 1.2, which are parallel to each other and to the longitudinal axis of the thigh and are joined together by means of an arch-shaped rigid or semirigid plate 1.3 and by straps 3.

The inner surface of the femoral plate 1.3 tracks the anterior part of the leg.

Linear openings 1.4 are present on each femoral arm 1.1, 1.2. The straps 3, which have a face made of nylon Velcro®, pass through such linear openings 1.4. The straps are wrapped all around the thigh anteriorly and posteriorly, thus securing the femoral support 1.1 to the thigh, together with the plate 1.3. The tibial support 2 consists of a lateral tibial arm 2.1 and a medial tibial arm 2.2, which are parallel to each other and to the longitudinal axis of the leg. Linear openings 2.3 are present on the tibial arms 2.1, 2.2 too. The straps 3, which have a face made of nylon Velcro®, pass through such linear openings 2.3, and are wrapped all around the leg, thus securing the tibial support 2 to the leg itself.

The distal ends of the femoral arms 1.1, 1.2 and the proximal ends of the tibial arms 2.1, 2.2 are joined together by means of two hinges, one placed laterally with respect to the knee, the other placed medially. The distal ends of each femoral arm 1.1, 1.2 and the proximal ends of the each tibial arm 2.1, 2.2, which make up the hinge, are shaped as plates and are rounded.

For clarity purposes, let us consider only the hinge present in the medial compartment of the knee: four pins 1.5, 1.6, 1.7, 1.8 are fixed on the rounded distal end of the medial femoral arm 1.2; two openings 1.9, 1.10 are also present on the same end of the medial femoral arm 1.2.

A first pin 1.5 is located on the distal end of the medial femoral arm 1.2 and is placed centrally along the “a” axis which coincides with the axis of longitudinal symmetry of the medial femoral arm 1.2 itself.

A second pin 1.6 is located on the distal end of the medial femoral arm 1.2 and is placed peripherally at a distance “I” with respect to the central pin 1.5, along the “b” axis, perpendicular to the “a” axis.

The axes “a” and “b” ideally divide the distal end of the medial femoral arm 1.2 into four quadrants: the first one oriented towards the foot and the posterior part of the leg; the second one oriented towards the foot and the anterior part of the leg; the third one oriented towards the root of the lower limb and the posterior part of the leg; the fourth one oriented towards the root of the lower limb and the anterior part of the leg.

The third pin 1.7 is placed proximally with respect to the afore-mentioned pins 1.5, 1.6, in the quadrant oriented towards the root of the lower limb and the posterior part of the leg. It is situated at a distance “r” with respect to the central pin 1.5. The “d” axis which passes through the centre of the proximal pin 1.7 and the centre of the central pin 1.5 deviates by a few degrees from the “a” axis. The peripheral edge of the proximal pin 1.7 is tangential to the “a” axis itself.

The “d” axis, which passes through the centre of the proximal pin 1.7 and the centre of the central pin 1.5, continues its path towards the peripheral distal edge of the medial femoral arm 1.2. Along such axis—at a distance “r” from the central pin 1.5—there is a fourth pin 1.8, diametrically opposed to the pin 1.7 vis-à-vis the central pin 1.5. The peripheral edge of the distal pin 1.8 too is tangential to the “a” axis. The distal pin 1.8 is situated in the quadrant oriented towards the foot and the anterior part of the leg.

The first opening 1.9 is located near the peripheral edge of the rounded distal end of the medial femoral arm 1.2 and is extended in the two quadrants oriented towards the posterior part of the leg for 130-140 degrees. The centre of a first semicircular extremity of the opening 1.9 is situated at a distance “r” from the central pin 1.5 along a “d1” axis which is perfectly symmetrical to the “d” axis vis-à-vis the “a” axis. The edge of the first semicircular extremity of the opening 1.9 is tangential to the “a” axis and to the peripheral edge of the distal pin 1.8. The opening 1.9 has a particular shape: over the first 25-30 degrees starting from the first extremity, it describes an arc of circumference; from 25-30 degrees on, up to 130-140 degrees, it turns into a spiral which gets near the rounded distal end of the medial femoral arm 1.2. More specifically, the points that make up the longitudinal axis of this spiral are situated at an increasingly lower distance from the centre of the rounded distal end of the medial femoral arm 1.2 as they get closer to the second extremity, which is semicircular too.

The second opening 1.10, diametrically opposed to the opening 1.9 with respect to the “a” axis, is located in the two quadrants oriented towards the anterior part of the leg. The centre of a first semicircular extremity of the opening 1.10 is situated at a distance “r” from the central pin 1.5 along the “d1” axis. The edge of the first semicircular extremity of the opening 1.10 is tangential to the “a” axis and to the peripheral edge of the proximal pin 1.7. The opening 1.10 too describes an arc of circumference over the first 25-30 degrees starting from the first extremity. Yet—contrary to the opening 1.9—from 25-30 degrees on, up to 130-140 degrees, it turns into a spiral which gets away from the centre of the rounded distal end of the medial femoral arm 1.2. More specifically, the points that make up the longitudinal axis of this spiral are situated at an increasingly greater distance from the centre of the rounded distal end as they get closer to the second extremity.

The plate located at the proximal end of the medial tibial arm 2.2 is provided with three openings 2.4, 2.5, 2.6 and two pins 2.7, 2.8.

The first tibial opening 2.4 has a rectangular shape and rounded extremities. The first tibial opening 2.4 is obtained by creating a hole at the centre of the plate of the proximal end of the medial tibial arm 2.2 and then proceeding towards the foot along the “a1” axis of longitudinal symmetry of the medial tibial arm 2.2. The hole is the proximal extremity of the first tibial opening 2.4.

The second opening 2.5 is located peripherally on the proximal end of the medial tibial arm 2.2 and is extended for 130°-140°. The first extremity of this second opening 2.5 is situated on a “b1” axis, perpendicular to the “a1” axis, at a distance “I” from the centre of the above-mentioned central hole from which the first opening 2.4 originates towards the anterior part of the leg. The other extremity of the peripheral opening 2.5 is placed at 130-140 degrees with respect to the “b1” axis. The peripheral opening 2.5 has a particular shape: over the first 25-30 degrees starting from the afore-mentioned “b1” axis, it describes a circumference whose centre coincides with that of the hole generating the central opening and whose radius is equal to “I”.

Subsequently, for the remaining 105-110 degrees, it turns into a spiral which returns towards the centre of the plate. The longitudinal axis of such spiral is obtained as the sequence of points of the end of a segment with a length “I” whose second end proceeds along the longitudinal axis of the central opening 2.4 (from the centre of the plate to the periphery).

The “a1” and “b1” axes ideally divide the proximal end of each medial tibial arm 2.2 into four quadrants; the first one is oriented towards the foot and the posterior part of the leg; the second one is oriented towards the foot and the anterior part of the leg; the third one is oriented towards the root of the lower limb and the posterior part of the leg; the fourth one is oriented towards the root of the lower limb and the anterior part of the leg.

The central opening 2.4 is placed in the two quadrants oriented towards the foot. The first peripheral opening 2.5 is placed in the two quadrants oriented towards the root of the lower limb.

The centre of a first semicircular extremity of the third opening 2.6, which is extended for 130-140 degrees, is placed at a distance “r” from the intersection of the “a1”, “b1” axes in the quadrant oriented towards the foot and the anterior part of the leg, and deviates by a few degrees from the “a1” axis. The edge of the extremity of the opening 2.6 is tangential to the “a1” axis. Over the first 25-30 degrees, the opening 2.6 describes an arc of circumference, while from 25-30 degrees up to 130-140 degrees, it turns into a spiral which gets near the centre of the rounded proximal end of the medial tibial arm 2.2. More specifically, the points that make up the longitudinal axis of such spiral are at an increasingly lower distance from the centre of the rounded proximal end as they get near the second extremity.

The first tibial pin 2.7 is situated distally vis-à-vis the central opening 2.4, at a distance “r” from the intersection of the “a1”, “b1” axes, in the quadrant oriented towards the foot and the posterior part of the leg. The “d2” axis which passes through the centre of the pin 2.7 and the intersection of the “a1”, “b1” axes deviates by a few degrees from the “a1” axis. The peripheral edge of the first tibial pin 2.7 is tangential to the “a1” axis and to the first extremity of the second peripheral opening 2.6.

This “d2” axis continues towards the proximal peripheral edge of the medial tibial arm 2.2. The second pin 2.8 is placed on such axis, at a distance “r” with respect to the intersection of the “a1”, “b1” axes. The pin 2.8 is diametrically opposed to the pin 2.7 with respect to the “a1” axis; consequently the “d2” axis which passes through the centre of the pin 2.8 and the intersection of the “a1”, “b1” axes deviates by a few degrees from the “a1” axis, though in the opposite direction. The peripheral edge of the pin 2.8 too is tangential to the “a1” axis. The pin 2.8 is placed in the quadrant oriented towards the root of the lower limb and the anterior part of the leg.

An extremity of both peripheral openings 2.5 of the tibial arms 2.1, 2.2 is situated on the “b1” axis, perpendicular to the “a1” axis. The two peripheral openings 2.5 extend towards the anterior part of the knee, in perfect symmetry to the knee itself. Similarly, the two peripheral openings 2.6 of the tibial arms 2.1, 2.2 extend towards the anterior part of the knee, in perfect symmetry to the knee itself. Similarly, the pins 2.7, 2.8 of the lateral tibial arm 2.1 are perfectly symmetrical to the pins 2.7, 2.8 of the medial tibial arm 2.2.

Similarly, the openings 1.9, 1.10 and the pins 1.6, 1.7, 1.8 of the lateral femoral arm 1.1 are perfectly symmetrical to the openings 1.9, 1.10 and to the pins 1.6, 1.7, 1.8 of the medial femoral arm 1.2.

The pins 1.5, 1.6, 1.7, 1.8, present on the medial femoral arm 1.2, are oriented towards the rounded proximal end of the medial tibial arm 2.2, whereas the pins 2.7, 2.8, present on the medial tibial arm 2.2 are oriented towards the rounded distal end of the medial femoral arm 1.2.

The central pin 1.5 of the medial femoral arm 1.2 is engaged in the central opening 2.4 of the medial tibial arm 2.2; the pin 1.6 of the medial femoral arm 1.2 is engaged in the first peripheral opening 2.5 of the medial tibial arm 2.2; the distal pin 1.8 of the medial femoral arm 1.2 is engaged in the second peripheral opening 2.6 of the medial tibial arm 2.2; the distal pin 2.7 of the medial tibial arm 2.2 is engaged in the opening 1.9 of the medial femoral arm 1.2; the proximal pin 2.8 of the medial tibial arm 2.2 is engaged in the opening 1.10 of the medial femoral arm 1.2.

The peripheral edge of the end of the medial tibial arm 2.2, which extends in the quadrants oriented towards the posterior part of the leg, has a particular shape: the initial section of the peripheral edge 2.9—extending in the posterior quadrant oriented towards the root of the limb—describes a circumference arc. The successive section of the peripheral edge 2.10—extending in the posterior quadrant oriented towards the foot—is shaped as a spiral that tends to get away from the centre of the plate. More specifically, the points which make up the longitudinal axis of such spiral are situated at an increasingly greater distance from the centre of the rounded proximal end of the medial tibial arm 2.2 as they get near the second extremity. The peripheral surface of the proximal pin 1.7 of the medial femoral arm 1.2 remains in constant contact with the peripheral edge 2.9, 2.10 of the medial tibial arm 2.2 during the roto-traslatory movement of the latter vis-à-vis the medial femoral arm 1.2.

The pins 1.5, 1.6, 1.7, 1.8 and the openings 1.9, 1.10 present on the rounded distal end of the medial femoral arm 1.2, the openings 2.4, 2.5, 2.6 and the pins 2.7, 2.8 present on the rounded distal end of the medial tibial arm 2.2 make up the medial hinge of the device. Similarly, the pins 1.5, 1.6, 1.7, 1.8 and the openings 1.9, 1.10 present on the rounded distal end of the lateral femoral arm 1.1, the openings 2.4, 2.5, 2.6 and the pins 2.7, 2.8 present on the rounded distal end of the lateral tibial arm 2.1 make up the lateral hinge of the device.

The longitudinal axis of the central pin 1.5 of the lateral hinge and the longitudinal axis of the central pin 1.5 of the medial hinge are coaxial. Such axis coincides with the “c” axis which passes through the femoral condyles and around which the rotation of the knee takes place.

The pins 1.5, 1.6, 1.7, 1.8 are each provided with a threaded hole along their longitudinal axis.

The external part of the hinge has a plate 4 with holes that are coaxial with the inner longitudinal holes of the pins 1.5, 1.6, 1.7, 1.8 of the medial femoral arm 1.2. The holes of the plate 4 accommodate the screws that engage the threaded holes of the pins 1.5, 1.6, 1.7, 1.8, thus securing the plate 4 to the medial femoral arm 1.2 and preventing disjunction of the medial tibial arm 2.2 from the medial femoral arm 1.2. The height of the pins 1.5, 1.6, 1.7, 1.8 is greater than the thickness of the medial tibial arm 2.2, which can therefore move freely between the plate 4 and the medial femoral arm 1.2.

Considering that the pins 2.7, 2.8 of the tibial arms 2.1, 2.2 and the pins 1.6, 1.7, 1.8 of the femoral arms 1.1, 1.2 are perfectly symmetrical, the plates 4 of the two hinges have perfectly symmetrical holes too.

As regards the movement in detail, the starting point is the alignment between the medial femoral arm 1.2 and the tibial arm 2.2. Under this condition, the “a” axis of symmetry of the medial femoral arm 1.2 is aligned with the “a1” axis of symmetry of the tibial arm 2.2.

During the first phase of flexion of the leg on the knee, from 0° to 30°:

the central pin 1.5 of the medial femoral arm 1.2 rotates in the proximal extremity of the central opening 2.4 of the tibial arm 2.2 by the hole placed at the centre of the rounded proximal end of the medial tibial arm 2.2;

the pin 1.6 of the medial femoral arm 1.2 slides in the circular part of the opening 2.5 of the medial tibial arm 2.2;

the proximal pin 1.7 of the medial femoral arm 1.2 slides on the circular peripheral edge 2.9 of the medial tibial arm 2.2;

the distal pin 1.8 of the medial femoral arm 1.2 slides in the circular part of the opening 2.6 of the medial tibial arm 2.2;

the distal pin 2.7 of the medial tibial arm 2.2 slides in the circular part of the opening 1.9 of the medial femoral arm 1.2;

the proximal pin 2.8 of the medial tibial arm 2.2 slides in the circular part of the peripheral opening 1.10 of the medial femoral arm 1.2.

In this phase of movement, the hinge performs a rotation movement similar to that of the knee during the first 30 degrees of flexion, which is generally the phase when the foot is fully on the ground.

The subject's bodyweight, which in this phase rests totally on the lower limb, is transmitted, by means of the lateral 1.1 and medial 1.2 femoral arms (secured to the thigh), to the lateral 2.1 and medial 2.2 tibial arms (secured to the leg). In particular, the bodyweight bears on the pins 1.7, 1.8 placed at the distal end of the femoral arms 1.1, 1.2 and on the pins 2.7, 2.8 placed at the proximal end of the tibial arms 2.1, 2.2.

As already pointed out, the knee joint has only two contact points: one between the femoral condyle and the tibial tray in the medial compartment (point A) and the other between the femoral condyle and the tibial tray in the lateral compartment (point B).

Thanks to the device in question, the bodyweight is distributed not only to the physiological contact points A and B, but also to the four pins of the medial hinge and to the four pins of the lateral hinge, placed on both sides of the knee itself. The four pins of each hinge transmit the load at their points of contact with the respective opening 2.6, 1.9, 1.10 and the edge 2.9.

Throughout the various phases of the movement, the specific position of each individual point of contact between pin and opening ensures the suppression of any rotation determined by the distance between the pin and the centre of the hinge.

In case of poor stability, the knee does not usually stick to a circular trajectory, thus modifying the relations between the articulation surfaces, and in particular, the vertical distance. Thanks to the device in question, a circular trajectory is preserved over the first 30 degrees, thereby avoiding a pathological overload of the knee, produced by the subject's bodyweight.

In the second phase of flexion of the leg on the thigh, from 30° to 135°:

the central pin 1.5 of the medial femoral arm 1.2 slides along the central opening 2.4 of the medial tibial arm 2.2 and reaches its distal extremity;

the pin 1.6 of the medial femoral arm 1.2 slides in the spiral section of the opening 2.5 of the medial tibial arm 2.2;

the proximal pin 1.7 of the medial femoral arm 1.2 slides on the spiral peripheral edge 2.10 of the medial tibial arm 2.2;

the distal pin 1.8 of the medial femoral arm 1.2 slides in the spiral section of the opening 2.6 of the medial tibial arm 2.2;

the distal pin 2.7 of the medial tibial arm 2.2 slides in the spiral section of the opening 1.9 of the medial femoral arm 1.2;

the proximal pin 2.8 of the medial tibial arm 2.2 slides in the spiral section of the peripheral opening 1.10 of the medial femoral arm 1.2.

In this phase of movement, the hinge performs a rotatory motion associated with an increasingly progressive sliding, similarly to what happens between the articulation surfaces, which—in a healthy knee—are kept in their proper relations by the cruciate ligaments. There is generally no knee overload in this phase of the movement.

In case of poor stability, however, the knee does not usually stick to a roto-traslatory trajectory, thus modifying the relations between the articulation surfaces. Particularly important to this end is therefore the guiding function imposed to the joint by the two hinges of the device through the pins 1.7, 1.8 placed at the distal end of the femoral arms 1.1, 1.2 and the pins 2.7, 2.8 placed at the proximal end of the tibial arms 2.1, 2.2.

In case of deficient anterior cruciate ligament, leading to the anterior sliding of the tibia (or posterior gliding of the femoral condyle) during flexo-extension, the pins 1.7, 2.7 can be removed, thereby generating a force component of the anterior pins 1.8, 2.8 which—by sliding in their respective openings 2.6, 1.10—tend to break down the load bearing on the joint into a force component directed towards the centre of the hinge, thus offsetting the anterior sliding of the tibial trays. In this case the inner surface of the femoral plate 1.3 tracks the posterior part of the leg.

In case of deficient posterior cruciate ligament, leading to the posterior sliding of the tibia (or anterior gliding of the femoral condyle) during flexo-extension, the anterior pins 1.8, 2.8 can be removed, thereby generating a force component of the posterior pins 1.7, 2.7 which—by sliding in the opening 1.9 and along the edge 2.9, 2.10—tend to break down the load bearing on the joint into a force component directed towards the centre of the hinge, thus offsetting the posterior sliding of the tibial trays.

In a second embodiment of the present invention, the medial femoral arm 1.2 is slightly longer than the lateral femoral arm 1.1.

The tibial support 2 consists of a lateral tibial arm 2.1 and a medial tibial arm 2.2, which are parallel to one another and to the longitudinal axis of the leg. They are joined together, however, by means of an arch-shaped tibial plate 2.11 made of harmonic steel. The tibial plate 2.11, placed centrally with respect to the tibial support 2, tracks the anterior part of the leg. The symmetry axis of the tibial plate 2.11 is parallel to the symmetry axis of the femoral plate 1.3.

The medial tibial arm 2.2 consists of two parts: a fixed medial tibial arm 2.12 secured to the tibial plate 2.11 and a free-moving medial tibial arm 2.13 connected to the fixed one by means of a second medial hinge.

The free-moving medial tibial arm 2.13 consists of a rectilinear plate extended between the medial femoral arm 1.2 and the fixed medial tibial arm 2.12. The medial femoral arm 1.2 of the femoral support 1 is connected to the proximal end of the free-moving medial tibial arm 2.13 by means of the medial hinge described in the first embodiment of the present invention.

The end of the free-moving medial tibial arm 2.13 connected to the first medial hinge has the shape of a plate and accommodates the three afore-mentioned openings 2.4, 2.5, 2.6 as well as the two pins 2.7, 2.8. The pins 1.5, 1.6, 1.7, 1.8 and the openings 1.9, 1.10 present on the rounded distal end of the medial femoral arm 1.2, the openings 2.4, 2.5, 2.6 and the pins 2.7, 2.8 present on the rounded proximal end of the free-moving medial tibial arm 2.13 make up the medial hinge of the device. In this case too, the peripheral edge 2.9, 2.10 of the end of the free-moving medial tibial arm 2.13, which extends in the quadrants oriented towards the posterior part of the leg, has a particular shape. The peripheral surface of the proximal pin 1.7 of the medial femoral arm 1.2 remains in constant contact with the peripheral edge 2.9, 2.10 of the free-moving medial tibial arm 2.13 during the roto-traslatory movement of the latter with respect to the medial femoral arm 1.2. The distal end of the free-moving medial tibial arm 2.13 is rounded and has a hole 2.14 on the symmetry axis of the free-moving medial tibial arm 2.13 itself, which is engaged by a pin 2.15 situated at the intersection between the symmetry axis of the tibial plate 2.11 and the symmetry axis of the fixed medial tibial arm 2.12.

The plate present on the proximal end of the lateral tibial arm 2.1 accommodates the three afore-mentioned openings 2.4, 2.5 e 2.6. The two pins 2.7, 2.8 are also secured to the plate itself. The pins 1.5, 1.6, 1.7, 1.8 and the openings 1.9, 1.10 present on the rounded distal end of the lateral femoral arm 1.1, the openings 2.4, 2.5, 2.6 and the pins 2.7, 2.8 present on the rounded proximal end of the lateral tibial arm 2.1 make up the lateral hinge of the device.

There are, however, some differences between the two peripheral openings 2.5 situated respectively on the lateral tibial arm 2.1 of the tibial support 2 and on the free-moving medial tibial arm 2.13. The circular part of the peripheral opening 2.5 situated on the free-moving medial tibial arm 2.13 has a width of about 15°-20°, while the circular part of the opening 2.5 situated on the lateral tibial arm 2.1 has a width of about 25°-30°. The spiral-shaped part of the peripheral opening 2.5 of the free-moving medial tibial arm 2.13 extends for about 115°-120°, whereas that of the peripheral opening 2.5 situated on the lateral tibial arm 2.1 extends for about 105°-110°. Furthermore, the spiral-shaped part of the peripheral opening 2.5 of the lateral tibial arm 2.1 returns towards the centre of the plate to a greater extent than the spiral-shaped part of the peripheral opening 2.5 of the free-moving medial tibial arm 2.13. The exact extent of this return—to be arranged at the time of construction of the peripheral opening 2.5 of the free-moving medial tibial arm 2.13—can be established as a result of a radiological study or anthropometric evaluation.

Similarly, the opening 2.6 situated on the free-moving medial tibial arm 2.13 presents a different shape from that of the lateral tibial arm 2.1. The circular part of the peripheral opening 2.6 situated on the free-moving medial tibial arm 2.13 has a width of about 15°-20°, whereas the circular part of the opening 2.6 situated on the lateral tibial arm 2.1 has a width of about 25°-30°. The spiral-shaped part of the peripheral opening 2.6 of the free-moving medial tibial arm 2.13 extends for about 115°-120°, whereas that of the peripheral opening 2.6 situated on the lateral tibial arm 2.1 extends for about 105°-110°. Moreover, the spiral-shaped part of the peripheral opening 2.6 of the lateral tibial arm 2.1 returns towards the centre of the plate to a greater extent than the spiral-shaped part of the peripheral opening 2.6 of the free-moving medial tibial arm 2.13. The opening 2.4 of the lateral tibial arm 2.1 is more elongated than the opening of the free-moving medial tibial arm 2.13.

The central opening 2.4 of the plate of the free-moving medial tibial arm 2.13 is engaged by the central pin 1.5 of the medial femoral arm 1.2. The peripheral opening 2.5 of the plate of the free-moving medial tibial arm 2.13 is engaged by the peripheral pin 1.6 of the medial femoral arm 1.2. The peripheral opening 2.6 of the plate of the free-moving medial tibial arm 2.13 is engaged by the peripheral pin 1.8 of the medial femoral arm 1.2.

The openings 1.9 and 1.10 placed on the lateral femoral arm 1.1 are different from the openings 1.9 and 1.10 placed on the medial femoral arm 1.2. The circular part of the opening 1.9 situated on the lateral femoral arm 1.1 has a width of about 25°-30°, whereas the circular part of the opening 1.9 situated on the medial femoral arm 1.2 has a width of about 15°-20°. The spiral-shaped part of the opening 1.9 situated on the lateral femoral arm 1.1 extends for about 105°-110°, whereas the spiral-shaped part of the opening 1.9 situated on the medial femoral arm 1.2 extends for about 115°-120°.

The spiral-shaped part of the opening 1.9 situated on the medial femoral arm 1.2 returns to a lesser extent towards the centre of the plate with respect to the opening 1.9 situated on the lateral femoral arm 1.1. The spiral-shaped part of the opening 1.10 situated on the medial femoral arm 1.2 returns to a greater extent towards the centre of the plate with respect to the opening 1.10 situated on the lateral femoral arm 1.1. The arc-shaped peripheral edge 2.9 present on the end of the free-moving medial tibial arm 2.13 has a width of about 15-20°, whereas the one present on the lateral tibial arm 2.1 has a width of about 25-30°. The spiral-shaped peripheral edge 2.10 present on the end of the free-moving medial tibial arm 2.13 has a width of 115°-120°, whereas the one present on the lateral tibial arm 2.1 has a width of 105°-110°. The spiral-shaped part of the peripheral edge 2.10 situated on the free-moving medial tibial arm 2.13 returns to a greater extent towards the centre of the plate itself with respect to the spiral-shaped part of the peripheral edge 2.10 situated on the lateral tibial arm 2.1.

An arc-shaped opening 2.16, with rounded extremities, is placed on the symmetry axis of the free-moving medial tibial arm 2.13, in a proximal position vis-à-vis the hole 2.14. The centre of an extremity of the opening 2.16 is placed on the symmetry axis of the free-moving medial tibial arm 2.13. The opening 2.16 extends in anterior direction.

Such opening 2.16 is engaged by a second pin 2.17, which is secured to the fixed medial tibial arm 2.12 and is placed proximally with respect to the pin 2.15 on the symmetry axis of the fixed medial tibial arm 2.12 itself. The pins 2.15, 2.17 have each a threaded hole along their longitudinal axis.

The two due pins 2.15, 2.17 secured to the fixed medial tibial arm 2.12, the hole 2.14 and the arc-shaped opening 2.16 of the free-moving medial tibial arm 2.13 make up the second medial hinge.

The second medial hinge too presents externally a plate 5 with holes, which are coaxial with the inner longitudinal holes of the pins 2.15, 2.17 of the fixed medial tibial arm 2.12. The holes of the plate 5 accommodate the screws which then engage the threaded holes of the pins 2.15, 2.17, securing the plate 5 to the fixed medial tibial arm 2.12 and avoiding the disjunction of the free-moving medial tibial arm 2.13 from the fixed medial tibial arm 2.12. The height of the pins 2.15, 2.17 is greater than the thickness of the free-moving medial tibial arm 2.13, which can therefore move freely between the plate 5 and the fixed medial tibial arm 2.12.

The difference of shape between the openings 2.4, 2.5, 2.6 situated on the free-moving medial tibial arm 2.13 and those situated on the lateral tibial arm 2.1 determines a difference of trajectory between the free-moving medial tibial arm 2.13 and the lateral tibial arm 2.1. The lateral tibial arm 2.1 imposes its trajectory to the fixed medial tibial arm 2.12 to which it is connected by means of the tibial plate 2.11.

During the flexion from 0 to 15-20 degrees, the symmetry axis of the free-moving medial tibial arm 2.13 is coaxial to the symmetry axis of the fixed medial tibial arm 2.12. Both are parallel to the symmetry axis of the lateral tibial arm 2.1.

From 15-20° to 135° of flexion, the symmetry axis of free-moving medial tibial arm 2.13 changes its angle with respect to that of the symmetry axis of the fixed medial tibial arm 2.12. The free-moving medial tibial arm 2.13 starts a roto-traslatory trajectory, imposed by its openings 2.5, 2.6, while the fixed medial tibial arm 2.12 remains on a circular trajectory as the openings 2.5, 2.6 of the lateral tibial arm 2.1 are arc-shaped up to 30 degrees.

The fulcrum of such change of angle between the symmetry axes lies in the pin 2.15 of the fixed medial tibial arm 2.12, which rotates within the hole 2.14 of the free-moving medial tibial arm 2.13 in which it is engaged, whereas the pin 2.17 of the fixed medial tibial arm 2.12 slides in the opening 2.16 of the free-moving medial tibial arm 2.13.

Similarly to what happens in the human knee, the device in question can track the automatic longitudinal rotation of the tibia.

This physiological motion makes the leg and the respective foot rotate externally, thus modifying also the position of the longitudinal axes of the leg itself, which remain always parallel to one another.

The same parallel pattern between the lateral tibial arm 2.1 and the fixed medial tibial arm 2.12 is preserved by the device, which, however, can rotate during flexo-extension by an extent equal to the cosine of the angle formed between the symmetry axis del free-moving medial tibial arm 2.13 and the symmetry axis of the fixed medial tibial arm 2.12, multiplied by the length comprised between the intersection of the “a1”, “b1” axes lying on the free-moving medial tibial arm 2.13 and the centre of the hole 2.14, which is engaged by the pin 2.15, situated on the fixed medial tibial arm 2.12, in such a way that the symmetry axis of the fixed medial tibial arm 2.12 and the symmetry axis of the lateral tibial arm 2.1 are always perfectly superimposed to the respective longitudinal axes of the leg, to which they are secured.

The angle of longitudinal rotation can be modified by changing the distance between the intersection of the “a1”, “b1” axes lying on the free-moving medial tibial arm 2.13 and the centre of the pin 2.15 of the fixed medial tibial arm 2.12, which must be equal to the distance between the intersection of the “a1”, “b1” axes lying on the lateral tibial arm 2.1 and the point of intersection between the symmetry axis of the tibial plate 2.11 and the symmetry axis of the lateral tibial arm 2.1.

In order to improve the adjustment of the device, the distal end of the free-moving medial tibial arm 2.13 accommodate three holes 2.18 along its symmetry axis and three arc-shaped openings 2.19. The position of the free-moving medial tibial arm 2.13 can thus be adjusted with respect to the tibial plate 2.11 by changing the holes 2.18 and the openings 2.19 which are engaged by the pins 2.15, 2.17 of the fixed medial tibial arm 2.12.

In order to further increase the adjustment of the device in question, the lateral tibial arm 2.1 too consists of a proximal lateral tibial arm 2.20 and a distal lateral tibial arm 2.21. The proximal lateral tibial arm 2.20 has an elongated shape and the distal lateral tibial arm 2.21 is secured to the tibial plate 2.11. Three equidistant holes 2.22 are situated on the prolongation of the proximal lateral tibial arm 2.20 and along its symmetry axis. The distal lateral tibial arm 2.21 accommodates two threaded holes 2.23, coaxial to at least two holes 2.22 present on the proximal lateral tibial arm 2.20. Two screws pass through the holes 2.22 of the proximal lateral tibial arm 2.20 and engage the two threaded holes 2.23 of the distal lateral tibial arm 2.21. The position of the distal lateral arm 2.21 can thus be adjusted with respect to the tibial plate 2.11 by allowing the screws to engage at each time different holes 2.23 of the distal lateral tibial arm 2.21.

1-10. (canceled)

11. An ergonomic knee device comprising:
a femoral support adapted to be secured to a user's upper leg including a lateral femoral arm and a medial femoral arm which are adapted to be generally parallel to one another when placed on medial and lateral sides of the user's upper leg and which have respective rounded distal end portions which are adapted to extend along lateral and medial sides of a user's knee when the femoral support is secured to the user's upper leg, the lateral and medial femoral arms being adapted to be parallel to a longitudinal axis of the user's upper leg, and each of the lateral and medial femoral arms having a longitudinal axis of symmetry “a”;
a tibial support adapted to be secured to a user's lower leg below the user's knee including a lateral tibial arm having a rounded proximal end portion, a fixed medial tibial arm, and a free-moving medial tibial arm having a rounded proximal end portion, wherein the lateral tibial arm and the fixed medial tibial arm are adapted to be parallel to one another so as to be parallel to a longitudinal axis of the user's lower leg, and each of the lateral tibial arm and the free-moving tibial arm having a longitudinal axis of symmetry “a1”;
a plurality of straps adapted to secure the femoral and tibial supports to a user's leg, wherein the plurality of straps pass through openings in respective lateral and medial femoral arms, the lateral tibial arm, and the fixed medial tibial arm;
a first arc-shaped plate interconnecting the lateral and medial femoral arms, the first arc-shaped plate being adapted to extend about an anterior part of the user's upper leg;
a second arc-shaped plate interconnecting the lateral tibial arm and the fixed medial tibial arm, the second arc-shaped plate being adapted to extend about an anterior part of the user's lower leg, with a symmetry axis of the second arc-shaped plate being parallel to a symmetry axis of the first arc-shaped plate;
each of the rounded distal end portions of the femoral support including first hinge elements which interact with second hinge elements of the rounded proximal end portions of each of the lateral tibial arm and the free-moving tibia arm;
the first hinge elements including a plurality of pins extending laterally outward of the distal femoral end portions and a pair of oppositely oriented arcuate slots in each distal femoral end portion which are spaced on opposition sides of the axis “a” of each of the lateral and medial femoral support arms, a first pin being positioned at each of the distal end portions along each axis “a” and forming two pivot axes of the first and second hinge elements relative to one another when assembled to one another, a second pin spaced between the first pin along an axis “b” which extends perpendicular to and intersecting the axis “a” at the first pin, and third proximal and fourth distal pins spaced adjacent to but on opposite sides of the axis “a” and oppositely spaced along an imaginary circle extending through both the third proximal pin and the pivot axes; the first, second, third and fourth pins and the arcuate slots of the distal femoral end portions of the first hinge elements being symmetrical with one another;
a first of the arcuate slots of the first hinge elements extend from first ends adjacent the third proximal pins and from adjacent the axis “a” generally along the imaginary circle and beyond the second pin toward the fourth pin with an outer portion of the first slots flaring outwardly relative to the imaginary circles, a second of the arcuate slots extending from first ends adjacent the fourth distal pins and from adjacent the axis “a” generally along the imaginary circles and having end portions that flare inwardly relative to the imaginary circles spaced from the third proximate pins;
the second hinge elements including first and second tibial pins extending laterally inward of the rounded proximal end portions of each of the lateral tibial arm and the free-moving tibial arm and wherein the first and second tibial pins of the lateral tibial arm are symmetrical to the first and second tibial pins of the free-moving medial tibial arm so as to be adjacent to but on opposite sides of an axes “a₁” of each of the lateral tibial and free-moving tibial arms and such that the first tibial pins are aligned to be movable within the first slots of the first hinge elements and the second tibial pins are aligned to be movable within the second slots of the first hinge elements;
the second hinge elements including first, second and third arcuate slots which are spaced from one another in the rounded proximal end portions of each of the lateral tibial arm and the free-moving tibial arm, the first arcuate slots extend generally linearly along the axes “a1” from a center point of each of the rounded end portions of the lateral tibial arm and the free-moving medial tibial arm toward distal ends of the rounded end portions and along the axes “a1”, each of the second arcuate slots extends in an arc spaced proximal of the first arcuate slots from a first end positioned at an axis “b1”, which extends perpendicular to and through axis “a1” at the center pivot point of the first arcuate slot, to an opposite end which extends to an opposite side and beyond the axis “a1” and between the first arcuate slot and the second tibial pins of the second hinge elements, the third arcuate slots extend in an arc from a first end adjacent the axis “a1” and radially outwardly and partially beyond the first end of the second arcuate slots of the second hinge elements along an arc located along an imaginary circle taken generally between the first and second pins of the second hinge elements;
The first pins of the first hinge elements forming the pivot axes of both the first and second hinge elements being mounted within the first arcuate slots of the second hinge elements and the second pins of the first hinge elements being mounted within the second arcuate slots of the second hinge elements and the third pins of the first hinge elements being slidable along an outer proximal arcuate edge portion of the rounded end portions of the lateral tibial arm and the free-moving media tibial arm from adjacent the second pins of the second hinge elements;
first and second opposing plates for covering the first and second hinge elements of the rounded distal end portions of the lateral femoral arm and medial femoral arm and the rounded proximal end portions of the lateral tibial arm and the free-moving medial tibial arm, the first and second plates having openings that align with the plurality of pins of the femoral support and tibial support, and fasteners for securing the first opposing plates to the plurality of pins of the first and second hinge elements to thereby prevent separation of the first and second hinge elements of the femoral support and the tibial support; and
the free-moving tibial arm having a distal end portion adapted to be connected by third hinge elements to a proximal end portion of the fixed medial tibial arm, wherein a third plate includes openings for receiving pins of the third hinge elements, and fasteners for connecting the third plate to the pins of the third hinge elements to thereby prevent separation of the free-moving tibial arm from the fixed medial tibial arm.

12. An ergonomic knee device, in accordance with claim 11, wherein the first end of the first arcuate slot of the lateral femoral arm forms an arc of a circle that extends between 25-30 degrees with the remaining portion of the first arcuate slot of the lateral femoral arm extending an additional 105-110 degrees, the first end of the first arcuate slot situated on the medial femoral arm forms an arc of a circle that extends between 15-20 degrees, the remaining portion of the first arcuate slot of the medial femoral arm extending for an additional 115-120 degrees, the remaining portion of the first arcuate slot of the lateral femoral arm arcing towards the center of the rounded distal end portion to a greater extent compared to the remaining portion of the first arcuate slot of the medial femoral arm, a second arcuate slot situated on the lateral femoral arm forming an arc of a circle that extends from a first end between 25-30 degrees with the remaining portion of the second arcuate slot extending an additional 105-110 degrees, a first end of the second arcuate slot situated on the medial femoral arm forming an arc of a circle extending between 15-20 degrees with the remaining portion of the second slot extending an additional 115-120 degrees, and the remaining portion of the second arcuate slot of the lateral femoral arm arcing towards the center of the rounded distal end portion to a lesser extent compared to the remaining end portion of the second arcuate slot of the medial femoral arm.

13. An ergonomic knee device, in accordance with claim 11, wherein the first, second, third and fourth pins of each of the lateral and medial femoral arms and the first and second tibial pins of the fixed medial tibial arm and the free-moving tibial arm are each provided with a threaded hole along a longitudinal axis thereof, the openings of the first, second and third outer plates accommodating passage of screws that engage the threaded holes of the first, second, third and fourth pins of the lateral and medial femoral arms and first and second tibial pins of the fixed medial tibial arm and the free-moving tibial arm.

14. An ergonomic knee device, in accordance with claim 11, wherein the first, second, third and fourth pins of the rounded distal end portions of the lateral and medial femoral arms and the first and second tibial pins of the fixed medial tibial arm and the free-moving medial tibial arm are each provided with a hole along a longitudinal axis thereof, the holes of the first, second and third outer plates accommodating rivets each having a free end that engages the holes of the first, second, third and fourth pins of the lateral and medial femoral arms and first and second pins of the fixed medial tibial arm and the free-moving medial tibial arm.

15. An ergonomic knee device, in accordance with claim 11, wherein the first, second, third and fourth pins of the rounded distal end portions of the lateral and medial femoral arms and the first and second pins of the fixed medial tibial arm and the free-moving medial tibial arm are each provided with an external thread on a free end thereof, each one of said threads being engaged by a nut.

16. An ergonomic knee device, in accordance with the claim 11, wherein the first, second, third and fourth pins of the lateral and medial femoral arms and first and second pins of the fixed medial tibial arm and the free-moving medial tibial arm are removably mounted to the lateral and medial femoral arms and free-moving medial arms, respectively.

17. An ergonomic knee device, in accordance with the claim 11 wherein the first end of the second arcuate slot of the free-moving medial tibial arm extends in a form of an arc of a circle between 15-20 degrees with the remaining portion of the second arcuate slot extending between an additional 115-120 degrees, the first end of the second arcuate slot of the lateral tibial arm extending in an arc of a circle between 25-30 degrees with the remaining portion of the second arcuate slot extending an additional 105-110 degrees, the remaining portion of the second arcuate tibial slot of the lateral tibial arm extending towards the center point of the rounded proximal end portion to a greater extent compared to the remaining portion of the second arcuate tibial slot of the free-moving medial tibial arm, the first end of the third arcuate slot of the free-moving medial tibial arm extending in an arc of a circle between 15-20 degrees with the remaining portion of the third arcuate slot extending an additional 115-120 degrees, the third arcuate slot of the lateral tibial arm extending from a first end between 25-30 degrees, with the remaining portion of the third arcuate slot of the lateral tibial arm extending an additional 105-110 degrees, and the remaining portion of the third arcuate slot of the lateral tibial arm turning towards the center point of the rounded proximal end portion to a greater extent compared to the remaining portion of the third arcuate slot of the free-moving medial tibial arm.

18. An ergonomic knee device, in accordance with claim 11 wherein the first arcuate slots formed in each of the respective distal end portions of the lateral and medial femoral arms extend from the first ends thereof over a first 25-30 degrees in an arc of circumference and therefrom up to an additional 130-140 degrees to the outer portions thereof, and wherein the second arcuate slots extend from the first ends thereof for a first 25-30 degrees in an arc of circumference and therefrom up to an additional 130-140 degrees to the outer portions thereof, and wherein the first and second arcuate slots of the lateral femoral arm are symmetrical to the first and second arcuate slots of the medial femoral arm.

19. An ergonomic knee device, in accordance with claim 11 wherein with respect to the second and third arcuate slots formed in the rounded proximal end portions of each of the lateral tibial arm and free-moving medial tibial arm, each of the second and third arcuate slots extends 25-30 degrees from the first ends thereof with each of the second and third arcuate slots having a shape of an arc of circumference and extending therefrom up to a total of 130 to 140 degrees forming an arc which approaches the center point of the respective rounded proximal end portions.

20. An ergonomic knee device, in accordance with the claim 11 wherein the first end of the second arcuate slot of the free-moving medial tibial arm extends in an arc of a circle between 15-20 degrees with the remaining portion of the second arcuate slot extending an additional 115-120 degrees, the second arcuate slot of the lateral tibial arm having a first end extending in an arc of a circle between 25-30 degrees with the remaining portion of the second arcuate slot extending an additional 105-110 degrees, the remaining portion of the second arcuate slot of the lateral tibial arm extending towards the center point of the rounded proximal end portion to a greater extent compared to the remaining portion of the second arcuate slot of the free-moving medial tibial arm, the third arcuate slot of the free-moving medial tibial arm extending from the first end in an arc of a circle between 15-20 degrees with the remaining portion of the third arcuate slot extending between 115-120 degrees, the third arcuate slot of the lateral tibial arm having a first end extending between 25-30 degrees, with the remaining portion of the third arcuate slot of the lateral tibial arm extending an additional 105-110 degrees, and the remaining portion of the third arcuate slot of the lateral tibial arm turning towards the center point of the rounded proximal end portion to a greater extent compared to the remaining portion of the third arcuate alot of the free-moving medial tibial arm.

21. An ergonomic knee device, in accordance with claim 11 wherein the third hinge element includes a pair of spaced pins extending from the fixed medial tibial arm and positioned along the axis “a1”, a hole in the distal end portion of the free-moving tibial arm of a size to receive a distal one of the spaced pins and another arcuate slot having open portions extending above and below the axis “a1” and oriented in an anterior direction and positioned inwardly of the fixed medial tibial arm with respect to the hole, and a proximal one of the spaced pins being received within and movable along the another arcuate slot.
